(19) United States
(12) Patent Application Publication
(10) Pub. No.: US 2020/0124550 A1
(43) Pub. Date: Apr. 23, 2020

(54) INDUCTIVE CHARACTERIZATION OF A METAL OBJECT EMBEDDED IN CONCRETE AND RELATED DETECTION DEVICE

(71) Applicant: PROCEQ SA, Schwerzenbach (CH)

(72) Inventors: Daniel DEMUTH, Eglisau (CH); Ralph MENNICKE, Uster (CH); Teddy LOELIGER, Uster (CH); Nicola RAMAGNANO, Rapperswil (CH)

(73) Assignee: PROCEQ SA, Schwerzenbach (CH)

(21) Appl. No.: 16/624,392

(22) PCT Filed: Jun. 20, 2017
(86) PCT No.: PCT/CH2017/000060

§ 371 Date: Dec. 19, 2019

(51) Int. Cl. G01N 27/02; G01N 27/72

(52) U.S. Cl. CPC G01N 27/023; G01N 33/383; G01N 27/72

(57) ABSTRACT

In order to characterize electrically conducting and/or ferromagnetic objects, such as rebars, in concrete, a device is rolled along a surface of the sample. The device comprises e.g. two rows (10.1, 10.2) of partially overlapping sending coils (6) and receiving coils (7). Each pair of attributed sending and receiving coils (6, 7) is designed to have reduced mutual impedance in the absence of any electrically conducting and/or ferromagnetic object. The complex value, e.g. the phase and absolute value, of the mutual impedances are measured in order to determine a number of parameters (size, position and coverage of the object with concrete) of the objects.

### TECHNICAL FIELD

The invention relates to a device and methods for characterizing an electrically conducting and/or ferromagnetic object, in particular a metal object, in particular a rebar, embedded in concrete.

### BACKGROUND ART

WO 2014/107816 describes a technique for characterizing a metal object, in particular a rebar, embedded in concrete. It comprises an array of coils arranged in a matrix. This array is placed against an object to be investigated. The device is then operated to measure the inductance of each coil in order to obtain information on the inner structure of the concrete.

### DISCLOSURE OF THE INVENTION

The problem to be solved by the present invention is to provide a device and method of this type with high reliability.

This problem is solved by the device and methods of the independent claims.

Hence, in a first aspect, the invention relates to a device for characterizing an electrically conducting and/or ferromagnetic object, in particular a rebar, embedded in concrete by means of inductive measurements. The device comprises the following elements:
A housing: This is a frame or outer shell of the device.
A plurality of sending coils and a plurality of receiving coils: These coils are arranged in or on said housing in at least one row.

Advantageously, the device further comprises wheels arranged on the housing. The wheels are oriented to roll the housing over a sample's surface along a displacement direction. This displacement direction extends transversally, in particular perpendicularly, to the row, which allows scanning a large surface area with only one row, or a few rows, of coils.

Further, each receiving coil can be designed to overlap with at least one attributed sending coil. The overlap is such that the mutual impedance between the receiving coil and its attributed sending coil is zero in the absence of the object in the measuring range of the device. This design makes the device more sensitive to the presence of the objects to be detected.

Advantageously, each one of at least some of the receiving coils is attributed to and overlaps with at least two sending coils, thus reducing the number of receiving coils required for a given spatial resolution.

Similarly, each one of at least some of the sending coils is attributed to and overlaps with at least two receiving coils, thus reducing the number of sending coils required for a given spatial resolution.

In a particularly compact design, each row comprises, alternatingly and in overlapping manner, a plurality of the receiving coils and a plurality of the sending coils.

There can also be more than one row of coils, in particular at least a first and a second row extending parallel to each other. In this case, advantageously, each of the rows comprises, alternatingly and in overlapping manner, a plurality of the receiving coils and a plurality of said the coils.

In a particularly advantageous design, the coils in the rows are staggered in respect to each other, in the sense that, in a projection perpendicular to the rows, each coil of the first row is arranged in the center between two overlapping coils of the second row. Thus, the second row can carry out measurements in the gaps of the first row, thereby increasing the spatial resolution of the measurement while still being able to use fairly large coils whose fields reach deeply into the sample.

In a second aspect, the invention relates to a method for operating the device described here. This method comprises repetitive steps of:
Sending a current through at least one of the sending coils; and
Measuring the voltage induced by the current in at least one of the receiving coils.

These as well as, optionally, all other steps described in the following can be carried out by a control unit of the device. Hence, the invention also relates to a device having a control unit adapted to carry out these as well as, optionally, the other steps.

The current is advantageously an alternating current, i.e. a current changing its flow direction repetitively, i.e. the current is not a pulse. In particular, the current is a CW alternating current. In this context, “CW” stands for “Continuous Wave”, and a “CW alternating current” is an alternating current with a duration of at least five, in particular of least ten, current cycles.

Alternatively, the current is a pulsed current.

In yet another aspect, the invention relates to a method for characterizing an electrically conducting and/or ferromagnetic object, in particular a rebar, embedded in concrete by means of inductive measurements. The method comprises the following steps:
Measuring a complex value indicative of the mutual impedance between a sending coil and a receiving coil or indicative of a self-impedance of a combined sending and receiving coil.
Using said complex value for characterizing the object.

In this context, a complex value indicative of the impedance is any pair of numeric values, such as real value and imaginary value or amplitude A and phase P, that define the full complex value of the impedance.

Further, the step of “using said complex value” involves using both numeric values, such as the amplitude as well as the phase or the real value as well as the imaginary value, in the characterization of the object.

This aspect of the invention is based on the understanding that the phase P of the complex impedance is a parameter that is able to provide valuable information about the embedded object.

The method of the third aspect can be carried out with a device able to determine the mutual impedance between a sending coil and a receiving coil, such as the one described above. Or it can be carried out with a device where the sending and receiving coils are the same coil, e.g. like the one described in WO 2014/107816.

The method of the third aspect can be part of a method for operating the device according to the second aspect of the invention, but it can also be a stand-alone method used with a different type of device.

Advantageously, the method can be used to calculate at least one of the following parameters:

c: the coverage of the object by the concrete,

p: the position of said object within a plane parallel to a surface of said concrete,

d: the diameter of the object,

μ: the magnetic permeability of the object,

σ: the electrical conductivity of said object.

Such a calculation can be based on using a mathematical model relating the amplitude parameter A and the phase parameter P to the parameters c, p, d, μ, σ.

In this case, the magnetic permeability and/or said electrical conductivity a is of particular interest because they strongly affect the phase of the impedance. Hence:
In one advantageous embodiment, the method comprises the step of calculating the permeability μ and/or the electrical conductivity σ from the mathematical model. Since the permeability and conductivity strongly affect the phase parameter, one or both of them can be well estimated from a measurement that delivers such a phase of the impedance.
In another advantageous embodiment, the user can be queried to provide the permeability μ and/or the electrical conductivity σ. In this case, the mathematical model's degrees of freedom are reduced, which allows to derive at least one of the other parameters, such as coverage c and/or diameter d, with higher accuracy.

The method of the third aspect can e.g. be carried out by using the device of the first aspect for measuring the complex impedance.

### BRIEF DESCRIPTION OF THE DRAWINGS

The invention will be better understood and objects other than those set forth above will become apparent when consideration is given to the following detailed description thereof. This description makes reference to the annexed drawings, wherein:

FIG. 1 a device for characterizing electrically conducting and/or ferromagnetic objects in concrete,

FIG. 2 a schematic sectional view of the device,

FIG. 3 a view of the coil assembly of the device,

FIG. 4 a block diagram of some components of the device,

FIG. 5 a first embodiment of a sending coil and a receiving coil with zero mutual impedance,

FIG. 6 a second embodiment of a sending coil and two receiving coils with zero mutual impedance,

FIG. 7 a third embodiment of a sending coil and a receiving coil with zero mutual impedance and concentric geometry,

FIG. 8 a diagram of rebar diameter vs. phase,

FIG. 9 a diagram of rebar diameter vs. phase for different rebar magnetic permeabilities,

FIG. 10 a diagram of cover thickness vs. phase,

FIG. 11 a diagram of rebar position vs. impedance (absolute value), and

FIG. 12 a diagram of rebar position vs. phase.

### MODES FOR CARRYING OUT THE INVENTION

Device Design:

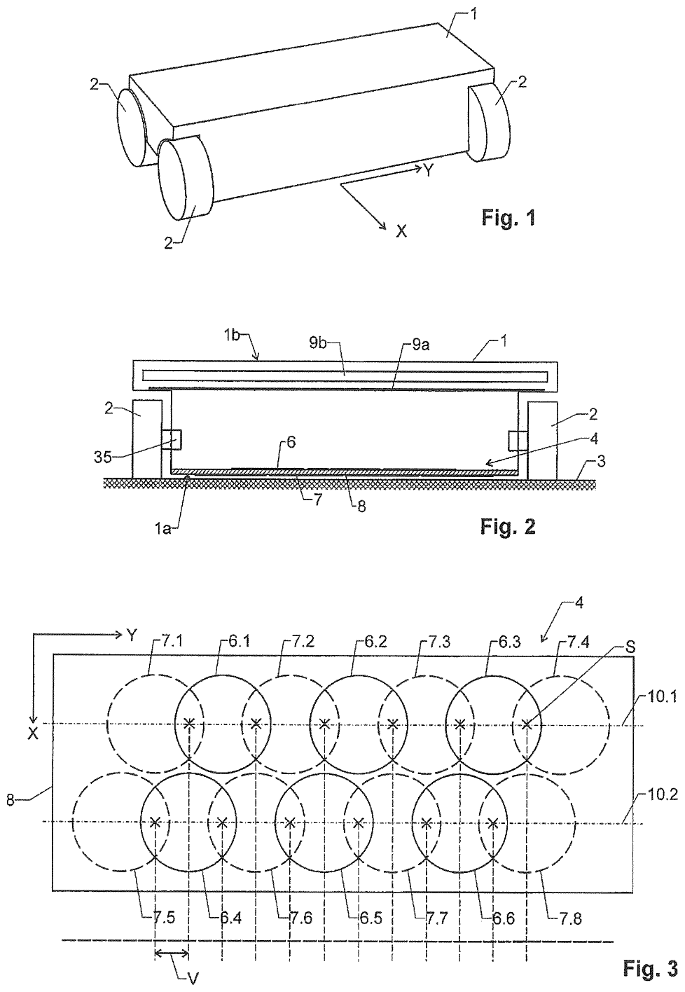

The device of FIGS. 1 and 2 comprises a housing 1 carrying electronics and sensors as described below. It is equipped with one or more wheels 2, by means of which it can be displaced along a direction X over a sample 3 of concrete to be tested.

As best seen in FIG. 2, the device comprises a coil assembly 4. When the wheels 2 are in contact with sample 3, coil assembly 4 is located on a side of the device facing the sample.

Coil assembly 4 comprises a number of sending coils 6 and a number of receiving coils 7. In the embodiment of FIG. 2, these coils are, by way of example, arranged on opposite sides of a carrier board 8, such as a printed circuit board, such that the sending coils 6 and the receiving coils 7 can be easily arranged in an overlapping manner.

Alternatively, the sending coils 6 and the receiving coils 7 can e.g. be implemented in different layers on the same side of a multi-layer printed circuit board.

FIG. 3 shows an example of an embodiment of coil assembly 4. In this example, the sending coils 6 (individually designated by reference numbers 6.1-6.6) are shown in solid lines, while the receiving coils 7 (designated as 7.1-7.8) are shown in dashed lines. Each coil is shown as a circle, but, in practice, it will be a coil of one or more windings, and it can have circular or non-circular, e.g. rectangular, design.

As mentioned, the coils 6, 7 can e.g. be implemented as metallic leads on a printed circuit board, even though discrete coils can be used as well.

FIG. 3 illustrates the overlapping design of the sending coils 6 and the receiving coils 7. As can be seen, in the shown embodiment, each sending coil 6 overlaps with two receiving coils 7, while each one but the peripheral ones of the receiving coils 7 overlaps with two sending coils 6.

As can be seen, only the peripheral receiving coils (namely coils 7.1, 7.4, 7.5 and 7.8) overlap with one sending coil only.

The region of overlap between a sending coil and a receiving coil corresponds to the location where the respective coil pair has highest sensitivity to electrically conducting and/or ferromagnetic objects embedded in sample 3.

As can further be seen from FIG. 3, the coils are arranged in two parallel rows 10.1, 10.2.

Each row 10.1, 10.2 consists, alternatingly and in overlapping manner, of a plurality of the receiving coils 7 and a plurality of the sending coils 6.

The coils of the two rows 10 are mutually offset, namely such that (in a projection along direction X) each coil of the first row 10.1 is arranged in the center between two overlapping coils of the second row 10.2, and vice versa. This allows to mutually stagger the regions of overlap of the two rows 10.1 and 10.2, thus increasing the spatial resolution along direction Y (perpendicular to direction X) as the device is moved along direction X over sample 3.

Each area where a sending coil 6 overlaps a receiving coil 7 forms an area of high sensitivity to the is objects to be detected. The point of highest sensitivity is located at the geometrical center (the centroid) of this region of overlap. This point is called the sensing node S. All sensing nodes of the embodiment of FIG. 3 are denoted by small crosses, one of which is tagged with S.

As shown in FIG. 3, the sensing nodes S are arranged along the two rows 10.1, and 10.2, but embodiments with a single row or more that two rows can be envisaged as well. The rows are parallel to each other.

The sensing nodes S of the individual rows 10.1, 10.2 are mutually offset by less than the distance between neighboring nodes S of the same row, namely such that (in a projection along direction X) they are arranged at regular intervals V along direction Y. Thus, an increased spatial resolution along direction Y (perpendicular to direction X) is achieved as the device is moved along direction X over sample 3.

As can be seen in FIG. 2, the coils 6, 7 are arranged at first side 1a of housing 1 of the device. This is the side that, in operation, faces sample 3. It is also the side of the wheels 2. Housing 1 further comprises a second side 1b opposite first side 1a.

The device of FIG. 2 further shows an electrically conducting and/or ferromagnetic shield 9a arranged at least at one side of the coils 6, 7. Shield 9a is positioned between the coils 6, 7 and second side 1b of housing 1, such that it shields the coils 6, 7 from any of the device's components, or external objects, located at the side of shield 9a that faces away from the coils 6, 7.

In particular, shield 9a is located between the coils 6, 7 and at least some of the electronic circuitry 9b of the device.

Advantageously, shield 9a is located at a distance from the coils 6, 7, in particular of a distance of at least 1 cm, in particular of at least 2 cm.

Shield 9a can be e.g. a ferrite (ferrimagnetic) sheet or any other electrically conducting and/or ferromagnetic material.

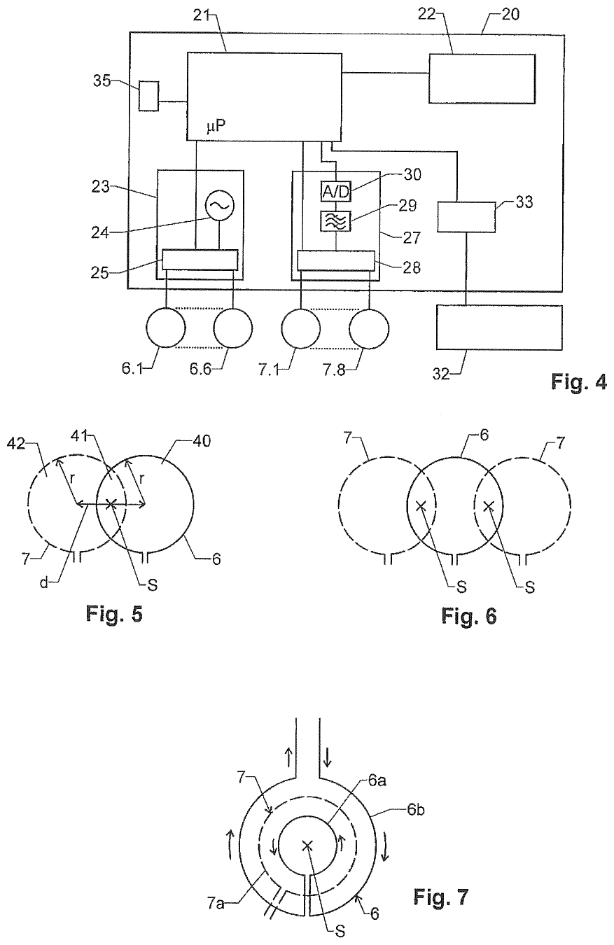

FIG. 4 shows some of the components of the device.

As can be seen, it includes a control unit 20 comprising e.g. a microprocessor 21 as well as a program and data memory 22.

Control unit 20 further comprises a driver 23 for selectively sending an alternating signal current and/or a pulsed current to each one of the sending coils 6.1-6.6.

More specifically, driver 23 is adapted to generate an alternating signal current or a pulsed current in one of the sending coils 6.1-6.6 at a time, and control unit 20 is adapted to subsequently send the signal current through each one of the sending coils 6.1-6.6.

For example, and as illustrated in FIG. 4, driver 23 can comprise an oscillator 24, whose output signal is sent to the input of a multiplexer 25. The sender coils are connected to the multiplexer's outputs. Control unit 20, e.g. microprocessor 21, controls multiplexer 25 to connect oscillator 24 to any of the sending coils 6.1-6.6.

It must be noted, though, that driver 23 can also be differently configured. For example, microprocessor 21 can be equipped with a sufficient numbers of outputs directly connected (optionally via amplifier circuitry and/or filter circuitry) to each of the sending coils 6.1-6.1 and it can be programmed to activate the desired output for generating the signal in the respective sending coil. Alternatively, part or all of the functionality of driver 23 can be implemented in an FPGA circuit.

Control unit 20 further comprises a receiver 27 for measuring the voltages induced in the receiving coils 7.1-7.8.

More specifically, receiver 27 is adapted to measure the voltage coupled into each receiving coil 7.1-7.8 when a signal current is sent through a sender coil that it overlaps with.

For example, and as illustrated in FIG. 4, receiver 27 can comprise a signal demultiplexer 28, whose inputs are connected to the receiving coils 7.1-7.8. Control unit 20, advantageously microprocessor 21, is connected to demultiplexer 28 for selecting which one of its inputs is connected to its output for further processing. The output of demultiplexer 28 can e.g. be fed to a filter and/or amplifier 29 and/or an analog-digital-converter 30.

Again, receiver 27 can also be differently configured. For example, most of its functionality can be implemented by the hard- and software of microprocessor 21, or it can be implemented as an FPGA, or additional filter and/or amplifier stages can be arranged between the receiving coils 7 and demultiplexer 28.

FIG. 4 further shows an input/output-device 32 connected to control unit 20 by means of a (wire-bound or wireless) interface 33. Input/output-device 32 allows the device to display its results and/or to query the user for input.

Input/output-device 32 can form part of the present device, or it may be implemented as separate, stand-alone equipment.

Finally, FIG. 4 as well as FIG. 2 show an encoder 35 capable to detect the rotation of at least one of the wheels 2. This allows to spatially correlate the measurements taken while the device is rolled over the sample along displacement direction X.

Operation:

In order to probe a given sample 3, the user first places the wheels 2 of the device on one of its surfaces, with coil assembly 4 facing the surface.

The device the starts a scanning operation, e.g. after manual or automatic triggering.

During scanning, control unit 20 subsequently sends the signal current through each one of the sending coils 6.1-6.1, e.g. in the order 6.1 . . . 6.6. Advantageously, in order to avoid undesired crosstalk, the signal current is sent only through a single one of the sending coils 6 at a time.

The signal current is an alternating current, advantageously of a single frequency, i.e. a sine-current, even though signals having harmonic spectral components or a superposition of multiple sine currents can be used as well.

Alternatively, the signal current is a pulsed current.

While sending the signal current through a given sending coil, e.g. sending coil 6.1, the voltages induced in its attributed, overlapping receiving coils (e.g. receiving coils 7.1 and 7.2) are measured. In other words, while sending the current through the single one sending coil, the induced voltages are measured in at least two receiving coils overlapping with the single one sending coil.

This measurement allows to determine the mutual impedance M between a sending coil and a receiving coil from

U=M·I

with U being the complex voltage in the receiving coil and I being the complex current in the sending coil. In case the mutual impedance M is purely inductive, the value M becomes purely imaginary and its value can be determined from

U=L·dI/dt (1)

with

M=i·ω·L

and with U being the voltage in the receiving coil and dI/dt being the time-derivative of the current in the sending coil.

Instead of measuring the impedance M explicitly, any other parameter indicative of it (i.e. any other parameter from which the impedance can be calculated) can be used.

In other words, the invention advantageously comprises the step of determining, from the measured voltage in the receiving coil, a measured parameter indicative of the mutual impedance of at least one of the sending coils and one of the receiving coils.

If the current I is a sine-signal, the current I and the voltage U can be expressed as complex numbers

I=I₀·exp(iωt) (2)

U=U₀·exp(iωt)

with ω designating the angular frequency of the current and I₀ and U₀ being complex numbers designating the amplitude and phase of the current and voltage, respectively. In this case, the mutual impedance M is also a complex number, and it can (ignoring the constant value of ω as well as a factor i), be defined as

M=U₀/I₀. (3)

In this case, mutual impedance M is a complex-valued quantity whose absolute value A describes the strength of the inductive coupling of the two coils and whose phase P describes the phase shift of the coupling, i.e.

M=A·exp(i·P) (4)

Equivalently, the complex mutual impedance M can e.g. be expressed by its real value Re(M) and its imaginary value Im(M).

The complex value of the mutual impedance or transimpedance M depends on the structure of the sample 3 at the region of the magnetic field extending between the sending and receiving coils. In particular, it is strongly influenced by any electrically conducting and/or ferromagnetic objects in this region.

Coil Design:

Advantageously, the geometry and mutual position of each pair of attributed sending and receiving coils 6, 7 is chosen such that, in the absence of electrically conducting and/or ferromagnetic objects in said region, the mutual impedance M is zero, in particular zero in the sense that it is at least five times smaller, in particular at least ten times smaller, in particular at least thirty times smaller, than the impedance of the self impedance of the sending coil and the receiving coil.

In the case of circular single-loop coils of equal radius r as shown in FIG. 3, and as shown in more detail in FIG. 5, a mutual impedance M of zero can be achieved if the distance D between the centers of the coils is in the order of e.g. 1.6·r, depending strongly on the number and distribution of the coil windings.

In more general terms, in this design the sending and receiving coils are arranged to cover several regions, namely:
A first region 40 covered by the sending coil only.
An overlap region 41 covered by both the sending and said receiving coil.
A second region 42 covered by the receiving coil only.

As mentioned above, the area of largest sensitivity of the coil design of FIG. 5 lies in the geometric center, i.e. at the centroid, of overlap region 41 where both the sending and receiving coils overlap. This point forms the sensing node S and is denoted by a small cross in FIG. 5.

As shown in FIGS. 3 and 6, the design of FIG. 5 can be used to build a row of overlapping sending and receiving coils 6, 7.

FIG. 7 shows another example of a design of the sending coil 6 and receiving coil 7 that has zero mutual impedance M in the absence of an electrically conducting and/or ferromagnetic object in the region of measurement.

In the embodiment shown, one of the two coils (e.g. the sending coil 6) forms a first current loop 6a and a second current loop 6b, with the first current loop 6a arranged within, in particular concentrically within, the second current loop 6b. The two current loops 6a, 6b are connected to carry opposite currents. The second coil (e.g. the receiving coil 7) forms a third current loop 7a arranged between the first and second current loops 6a, 6b.

In the embodiment of FIG. 7, the sensing node S is arranged in the center of the three current loops 6a, 7b, 7a.

The loops 6a, 6b, 7a can consist of single windings or multiple windings.

Calibration:

In order to improve signal accuracy, the device can be calibrated, and calibration data can be stored in memory 22.

For example, calibration measurements can be carried out in the absence of any electrically conducting and/or ferromagnetic object in the measuring range. For each pair of a sending coil 6 and an attributed receiving coil 7, the mutual impedance M0 is measured. Even though this mutual impedance M0 should be zero, in reality there will always be a small deviation from that value. The deviations for all such coil pairs are stored as calibration parameters in memory 22.

When carrying out a measurement, the measured parameter M (i.e. the parameter indicative of the mutual impedance between two attributed sending and receiving coils) is compared to the respective calibration parameter M0 of the same pair of coils, e.g. by calculating the difference M−M0, and using the result for characterizing the electrically conducting and/or ferromagnetic objects in sample 3.

Signal Processing:

As shown in Eq. (4), the complex mutual impedance M can e.g. be expressed by an amplitude parameter A as well as a phase parameter P.

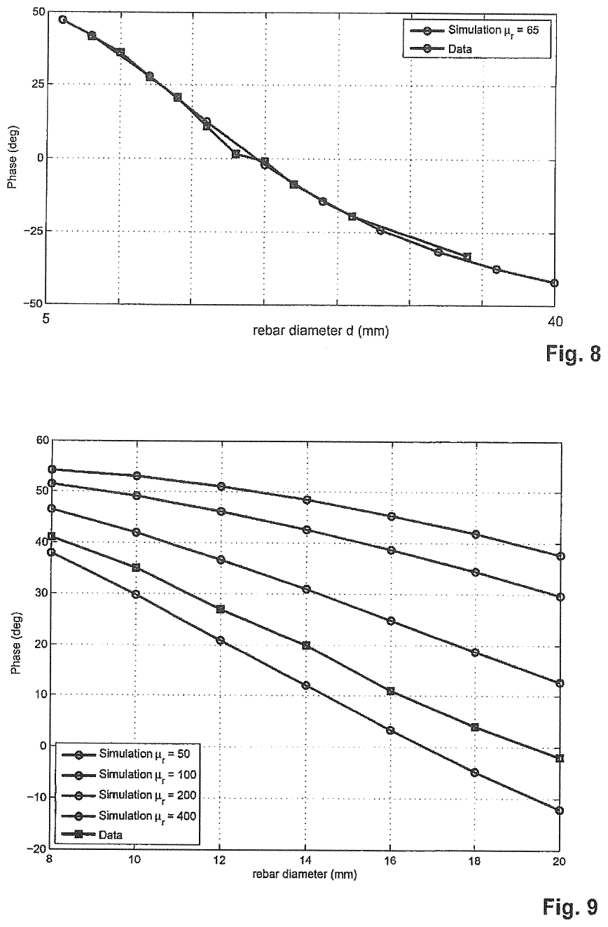

Advantageously, the present invention not only involves measuring the amplitude parameter A but also the phase parameter P (or, for example, the real and imaginary values of the complex impedance) because the phase information provides additional important insight for characterizing the electrically conducting and/or ferromagnetic objects embedded in sample 3. This is illustrated under reference to FIGS. 8-11.

FIG. 8 shows simulation data as well as measured data for the phase P as a function of the diameter of a rebar having a relative permeability μ=65 and being embedded under a coverage of c=50 mm in concrete.

As can be seen, the phase P depends strongly on the diameter d.

FIG. 9 shows that the phase P also depends on the permeability of the rebar.

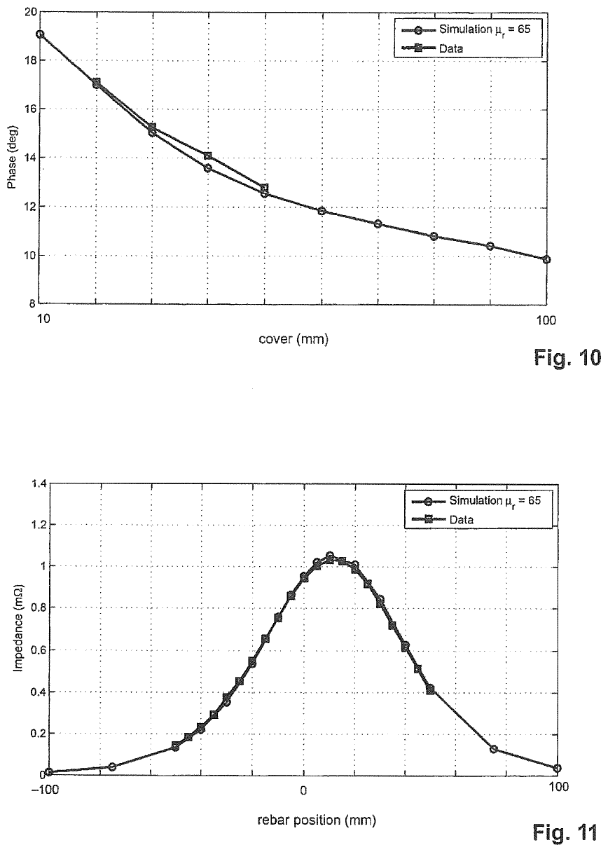

FIG. 10 shows, on the other hand, that the phase P changes only weakly with the cover depth c.

FIG. 11 shows the amplitude parameter A (the absolute value of the impedance) as a function of the rebar position (i.e. of the relative position, along direction X, of the center of the sending coil of FIG. 5 and the rebar). As can be seen, that value varies strongly with position, and the width of the peak is a function of the coverage c as well as the diameter d.

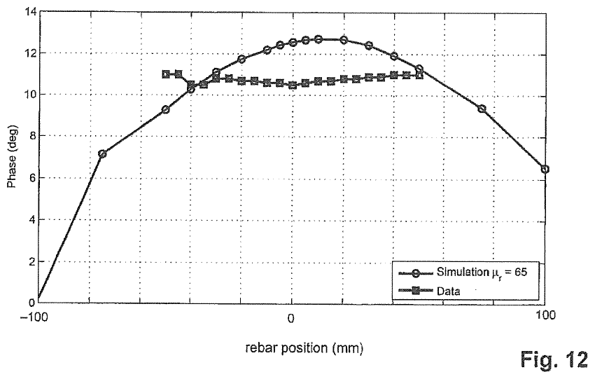

FIG. 12 shows the same dependence as FIG. 11 so but for the phase. As can be seen, the phase P varies less strongly.

FIGS. 9 to 12 illustrate that the behaviors of the phase P as well as the amplitude A are functions of the following parameters:

c: The coverage of the object, i.e. its depth within the concrete sample;

p: the position of said object within a plane parallel to a surface of the concrete;

d: The diameter of the object;

μ: The magnetic permeability of the object;

σ: the electrical conductivity of said object.

This dependence can be expressed in a mathematical model, i.e.

A=A(c,p,d,μ,σ)

P=P(c,p,d,μ,σ). (5)

Considering different frequencies f of the excitation current, this dependence can be expressed in a more sophisticated mathematical model, i.e.

A=A(c,p,d,μ,σ,f)

P=P(c,p,d,μ,σ,f). (6)

By using a frequency-dependent model as expressed by Eq. (6), additional information can be gained, in particular because the permeability μ as well as (less so) the conductivity σ are frequency-dependent parameters.

Hence, advantageously, the invention comprises the step of measuring the complex value describing the mutual impedance and/or self-impedance at a plurality of frequencies, e.g. by applying these frequencies sequentially, or by applying a superposition of these frequencies and filtering the returned signal.

The functions A and P can be calculated using simulation methods, such as finite element simulation, e.g. as provided by the Comsol Multiphysics® modeling software by Comsol Inc., Burlington, USA.

Hence, the knowledge of the measured values of A as well as P at certain frequencies f allows to determine at least one of the parameters c, p, d, a and using standard curve fitting techniques or equalization calculus.

In particular, as can be seen from the strong dependence in FIG. 9, a knowledge of the phase P allows to determine the magnetic permeability μ and/or conductivity σ with good accuracy.

On the other hand, if this permeability μ and/or conductivity σ is known, the method of measurement can comprise the step of querying the user to provide one or both of these values for the given object to be characterized, e.g. by entering it on input/output-device 32. This allows to determine the parameters c and/or d with greater accuracy.

By translating the device along direction X over sample 3 and continuously measuring the values of amplitude A and phase P as a function of the wheels' position, a spatially resolved image can be recorded.

Hence, the invention also relates to a method of operating the device, with said method comprising the step of displacing the device in a displacement direction X transversally to, in particular perpendicularly to, at least one row 10.1, 10.2 of coils while recording the spatial distribution of the object. The recording takes place in the direction Y of the row 10.1, 10.2 (by using the results of the different mutual impedances) as well as in the displacement direction X (by repeating the measurements while displacing the device along X).

This procedure allows e.g. detecting a rebar extending parallel to the displacement direction X. (Detecting this type of rebar is difficult or even impossible with conventional designs using only a single coil or a single pair of coils.)

The offset between the two rows 10.1 and 10.2 (FIG. 3) is taken into account for correlating the measurements of the coils in these rows.

The data recorded in this way can be used for generating a three-dimensional model of the electrically conducting and/or ferromagnetic objects using the techniques described by S. Quek et al. in NDT&E International 36(2003), 7-18.

Notes:

It must be noted, that the roles of the sending coils 6 and the receiving coils 7 can be swapped because the mutual impedance from a given coil to another coil is the same as the one from the other coil to the given coil. Hence, for example in FIG. 3, the coils 6.x can act as receiving coils and the coils 7.x can act as sending coils. Similarly, in FIGS. 5-7, the coils 7 can act as sending coils and the coils 6 can act as receiving coils.

While there are shown and described presently preferred embodiments of the invention, it is to be distinctly understood that the invention is not limited thereto but may be otherwise variously embodied and practiced within the scope of the following claims.

1. A device for characterizing an electrically conducting and/or ferromagnetic object, in particular a rebar, embedded in concrete by means of inductive measurements, said device comprising
a housing,
a plurality of sending coils and a plurality of receiving coils arranged in or on said housing in at least one row.

2. The device of claim 1 further comprising wheels arranged on said housing, wherein said wheels are oriented to roll said housing over a sample's surface along a displacement direction transversal to said row.

3. The device of claim 2 further comprising an encoder detecting a rotation of at least one of said wheels.

4. The device of claim 2 wherein said displacement direction is perpendicular to said row.

5. The device of claim 1 further comprising
a driver adapted and structured to generate an alternating current and/or a pulsed current in said sending coils, and
a receiver adapted and structured to measure a voltage induced by said current in said receiving coils.

6. The device of claim 1 wherein each receiving coil overlaps, in an overlap region, with at least one attributed sending coil, wherein a mutual impedance between said receiving coil and said attributed sending coil is zero in the absence of the object in a measuring range of said device.

7. The device of claim 6 wherein each one of at least some of the receiving coils is attributed to and overlaps with at least two sending coils.

8. The device of claim 6 wherein each one of at least some of the sending coils is attributed to and overlaps with at least two receiving coils.

9. The device of claim 7 wherein said receiving coil and its attributed sending coil cover
a first region covered by said sending coil only,
the overlap region covered by both said sending and said receiving coils, and
a second region covered by said receiving coil only.

10. The device of claim 7 wherein
a first one of said attributed receiving coil and sending coil forms a first current loop and a second current loop, with said first current loop arranged within, in particular concentrically within, said second current loop, and wherein said first and said second current loop are connected to carry opposite currents,
a second one said attributed receiving coil and sending coil forms a third current loop arranged between said first and said second current loop.

11. The device of claim 1, wherein each receiving coil overlaps, in an overlap region, with at least one attributed sending coil, wherein each overlap region defines a sensing node at a geometric center of said overlap region,
wherein said sensing nodes are arranged in several parallel rows and are mutually offset, by less than the distance between neighboring nodes of the same row, such that, in a projection along a direction perpendicular to said rows, the sensing nodes are arranged at regular intervals.

12. The device of claim 1 wherein said at least one row comprises, alternatingly and in overlapping manner, a plurality of said receiving coils and a plurality of said sending coils.

13. The device of claim 1 comprising at least a first and a second row of said coils, wherein said rows extend parallel to each other.

14. The device of claim 13 wherein each of said rows, alternatingly and in overlapping manner, a plurality of said receiving coils and a plurality of said sending coils.

15. The device of claim 14 wherein, in a projection perpendicular to said row, each coil of said first row is arranged in a center between two overlapping coils of said second row.

16. The device of claim 1 wherein said coils are arranged at a first side of said housing and wherein said housing further comprises a second side opposite to said first side, and wherein said device comprises
an electrically conducting and/or ferromagnetic shield positioned between said coils and said second side of said housing,
and in particular wherein said shield is located at a distance from the coils and/or wherein said shield is located between the coils and at least some electronic circuitry of said device.

17. A method for operating the device of claim 1 comprising repetitively
sending a current through at least one of said sending coils and
measuring a voltage induced by said current in at least one of said receiving coils.

18. The method of claim 17 wherein said current is an alternating current and/or a pulsed current, in particular a CW alternating current.

19. The method of claim 17 wherein said current is sent only through a single one of said sending coils at a time.

20. The method of claim 19 comprising, while sending said current through said single one sending coil, measuring the induced voltage in at least two receiving coils overlapping with said single one sending coil.

21. The method of claim 17 comprising determining, from said voltage, a measured parameter indicative of a mutual impedance of at least one of said sending coils and one of said receiving coils.

22. The method of claim 21 further comprising comparing said measured parameter to a calibration parameter indicative to the mutual impedance between said one of said sending coils and said one of said receiving coils in the absence of said object.

23. The method of claim 17, comprising displacing said device in a displacement direction transversally to, in particular perpendicularly to, said at least one row while recording a spatial distribution of said object in a direction of said at least one row as well as in said displacement direction.

24. A method, in particular of claim 17, for characterizing an electrically conducting and/or ferromagnetic object, in particular a rebar, embedded in concrete by inductive measurements, said method comprising
measuring a complex value indicative of a mutual impedance between a sending coil and a receiving coil or indicative of a self-impedance of a combined sending and receiving coil,
using said complex value for characterizing the object.

25. The method of claim 24 comprising calculating at least one of the parameters
c: a coverage of said object by said concrete,
p: a position of said object within a plane parallel to a surface of said concrete,
d: a diameter of said object,
μ: a magnetic permeability of said object,
σ: an electrical conductivity of said object,
using a mathematical model relating said amplitude parameter A and said phase parameter P to said parameters c, p, d, μ, σ.

26. The method of claim 25 comprising calculating said magnetic permeability μ and/or said electrical conductivity σ from said mathematical model.

27. The method of claim 25 comprising querying a user to provide said magnetic permeability μ and/or said electrical conductivity σ for a given object to be characterized.

28. The method of claim 24 comprising using a device for measuring said complex impedance.

29. The method of claim 24 comprising measuring said complex value at a plurality of frequencies.

30. A device for characterizing an electrically conducting and/or ferromagnetic object, in particular a rebar, embedded in concrete by means of inductive measurements, said device comprising
a housing,
a plurality of sending coils and a plurality of receiving coils arranged in or on said housing in at least one row,
wherein each receiving coil overlaps, in an overlap region, with at least one attributed sending coil, wherein each overlap region defines a sensing node at a geometric center of said overlap region,
wherein said sensing nodes are arranged in several parallel rows and are mutually offset, by less than the distance between neighboring nodes of the same row, such that, in a projection along a direction perpendicular to said rows, the sensing nodes are arranged at regular intervals.
